(19) United States
(12) Patent Application Publication
(10) Pub. No.: US 2008/0045450 A1
(43) Pub. Date: Feb. 21, 2008

(54) GLP-2 Derivatives

(76) Inventors: Liselotte Bjerre Knudsen, Valby (DK); Per Olaf Huusfeldt, Kobenhavn K (DK); Per Franklin Nielsen, Varlose (DK); Niels C. Kaarsholm, Vanlose (DK); Helle Birk Olsen, Allerod (DK); Lars Thim, Gentofte (DK); Soren Erik Bjorn, Lyngby (DK)

Correspondence Address: NOVO NORDISK, INC.;PATENT DEPARTMENT, 100 COLLEGE ROAD WEST, PRINCETON, NJ, 08540, US

(21) Appl. No.: 11/277,493

(22) Filed: Mar. 25, 2006

Related U.S. Application Data
(63) Continuation of application No. 10/730,215, filed on Dec. 8, 2003, now abandoned.
Application No. 10/730,215 is a continuation of application No. 09/908,534, filed on Jul. 18, 2001.
Application No. 09/908,534 is a continuation of application No. 09/258,187, filed on Feb. 25, 1999, now abandoned.
Application No. 09/258,187 is a continuation-in-part of application No. 08/922,200, filed on Sep. 2, 1997, now abandoned.
(60) Provisional application No. 60/035,905, filed on Jan. 24, 1997.
(60) Provisional application No. 60/036,226, filed on Jan. 25, 1997.

(30) Foreign Application Priority Data
Aug. 30, 1996 (DK) 0931/96
Nov. 8, 1996 (DK) 1259/96
Feb. 27, 1998 (DK) 0271/98

(51) Int. Cl. A61K 38/17; A61P 1/00; C07K 14/00

(52) U.S. Cl. 514/12; 530/324

(57) ABSTRACT

Analogs of GLP-2, pharmaceutical compositions comprising GLP-2 analogs, and methods of treating diseases and disorders comprising administering such analogs or compositions are provided.

### CROSS-REFERENCE TO RELATED APPLICATIONS

This application is a continuation of U.S. patent application Ser. No. 10/730,215 filed Dec. 8, 2003, which is a continuation of Ser. No. 09/908,534, filed Jul. 18, 2001, which is a continuation of Ser. No. 09/258,187 filed Feb. 25, 1999 which is a continuation-in-part of application Ser. No. 08/922,200, filed Sep. 2, 1997, which claims priority of Danish application serial nos. 0931/96, 1259/96 and 0271/98 filed Aug. 30, 1996, Nov. 8, 1996 and Feb. 27, 1998, respectively, and of U.S. Provisional Patent Applications 60/035,905, 60/036,226 and 60/085,789 filed Jan. 24, 1997, Jan. 24, 1997 and May 18, 1998, respectively, the contents of all of which are fully incorporated herein by reference.

### FIELD OF THE INVENTION

The present invention relates to derivatives of human glucagon-like peptide-2 (hGLP-2) and of analogues and/or fragments thereof which have a protracted profile of action and to methods of making and using them. The present invention also relates to pharmaceutical compositions comprising a GLP-2 derivative of improved solubility and/or stability, and to a method for improving the solubility and/or stability of GLP-2 or a fragment and/or analogue thereof.

### BACKGROUND OF THE INVENTION

Peptides are widely used in medical practice, and since they can be produced by recombinant DNA technology it can be expected that their importance will increase also in the years to come. When native peptides or analogues thereof are used in therapy it is generally found that they have a high clearance. A high clearance of a therapeutic agent is inconvenient in cases where it is desired to maintain a high blood level thereof over a prolonged period of time since repeated administrations will then be necessary. Examples of peptides which have a high clearance are: ACTH, corticotropin-releasing factor, angiotensin, calcitonin, insulin, glucagon, glucagon-like peptide-1, glucagon-like peptide-2, insulin-like growth factor-1, insulin-like growth factor-2, gastric inhibitory peptide, growth hormone-releasing factor, pituitary adenylate cyclase activating peptide, secretin, enterogastrin, somatostatin, somatotropin, somatomedin, parathyroid hormone, thrombopoietin, erythropoietin, hypothalamic releasing factors, prolactin, thyroid stimulating hormones, endorphins, enkephalins, vasopressin, oxytocin, opiods and analogues thereof, superoxide dismutase, interferon, asparaginase, arginase, arginine deaminase, adenosine deaminase and ribonuclease. In some cases it is possible to influence the release profile of peptides by applying suitable pharmaceutical compositions, but this approach has various shortcomings and is not generally applicable.

Preproglucagon, from which GLP-2 originates, is synthesized, inter alia, in the L-cells in the distal ileum, in the pancreas and in the brain. Processing of preproglucagon to give GLP-1 and GLP-2 occurs mainly in the L-cells. GLP-2 is a 33 amino acid residue peptide and possibly 34 amino acid residues in some tissue.

The amino acid sequence of GLP-2 and other preproglucagon fragments is given i.a. (“inter alia”) by Schmidt et al. (Diabetologia 28 704-707 (1985). Little is known about the physical chemical properties of GLP-2 but GLP-2 is expected, like GLP-1, to be a highly flexible and unstable molecule. GLP-2 and fragments and/or analogues thereof are potentially useful i.a. in regulation of appetite and in the treatment of small bowel syndrome. However, the high clearance limits the usefulness of these compounds, and thus there still is a need for improvements in this field.

It is an object of the present invention to provide improved GLP-2 compounds whose plasma profile is highly protracted while retaining activity.

It is another object of the present invention to provide pharmaceutical solutions comprising GLP-2 derivatives with improved solubility and stability.

### SUMMARY OF THE INVENTION

The present invention relates to derivatives of human glucagon-like peptide-2 (hGLP-2) and of analogues and/or fragments thereof which have a protracted profile of action and to methods of making and using them. The present invention also relates to pharmaceutical compositions comprising a GLP-2 derivative of improved solubility and/or stability, and to a method for improving the solubility and/or stability of GLP-2 or a fragment and/or analogue thereof.

The present invention also relates to a pharmaceutical composition comprising a GLP-2 derivative and a pharmaceutically acceptable vehicle or carrier.

The present invention also relates to the use of a GLP-2 derivative of the invention for the preparation of a medicament which has a more protracted action than the parent peptide.

The present invention also relates to the use of a GLP-2 derivative of the invention for the preparation of a medicament with protracted effect for the treatment of obesity.

The present invention also relates to the use of a GLP-2 derivative of the invention for the preparation of a medicament with protracted effect for the treatment of small bowel syndrome.

### DETAILED DESCRIPTION OF THE INVENTION

A simple system is used to describe fragments, analogues, and derivatives of GLP-2. For example, Lys²⁰GLP-2(1-33) designates a fragment of GLP-2 formally derived from GLP-2 by deleting the amino acid residues No. 34 and substituting the naturally occurring amino acid residue in position 20 (Arg) by Lys. Similarly, Arg³⁰Lys³⁵(N^{ε}-tetradecanoyl)GLP-1(1-35) designates a derivative of a GLP-2 analogue formally derived from GLP-2 by C-terminal addition of a Lys residue, exchange of the naturally occurring amino acid residue in position 30 (Lys) with an Arg residue and tetradecanoylation of the ε-amino group of the Lys residue in position 35.

Parent GLP-2 Peptide

The present invention relates to derivatives of GLP-2 and analogues and/or fragments thereof. The derivatives of the present invention have interesting pharmacological properties; in particular they have a more protracted profile of action than the parent peptides. Unless otherwise specified, “GLP-2” is defined herein as human GLP-2. The term “analogue” is defined herein as a peptide wherein one or more amino acid residues of the parent peptide have been substituted by another amino acid residue and/or wherein one or more amino acid residues of the parent peptide have been deleted and/or wherein one or more amino acid residues have been added to the parent peptide. Each mutation can take place either at any amino acid, including the N-terminal end or C-terminal amino acid. In a preferred embodiment, the parent GLP-2 peptide has a total of up to fifteen, preferably up to ten, more preferably up to six, amino acid residues have been exchanged with any ε-amino acid residue which can be coded for by the genetic code. In a further preferred embodiment, the parent GLP-2 peptide is human GLP-2 wherein a total of up to six, more preferably up to three, amino acid residues have been added, deleted or substituted with other amino acid residues which can be coded for by the genetic code.

In a preferred embodiment, the present invention relates to a GLP-2 derivative wherein the parent peptide has the following amino acid sequence (SEQ ID NO:1):

X¹ H X² D G S F S D E M N T X³ L D X⁴ L A X⁵ X⁶ D
F I N W L X⁷ X⁸ T K I T D X⁹

wherein
X¹ is NH₂, DFPEEVAIVEELGRR (SEQ ID NO:2), DFPEEVTIVEELGRR (SEQ ID NO:3), DFPEEVNIVEELRRR (SEQ ID NO:4), or a fragment thereof,
X² is Ala or Gly,
X³ is Ile or Val,
X⁴ is Asn, Ser or H is,
X⁵ is Ala or Thr,
X⁶ is Arg or Lys,
X⁷ is Ile or Leu,
X⁸ is Gln or H is, and
X⁹ is OH, Lys, Arg, Arg-Lys, Lys-Arg, Arg-Arg or Lys-Lys.

In a preferred embodiment, the parent peptide is GLP-2(1-30); GLP-2(1-31); GLP-2(1-32); GLP-2(1-33); GLP-2(1-34) or GLP-2(1-35). In another preferred embodiment, the parent peptide is:
Lys²⁰GLP-2(1-33);
Lys²⁰Arg³⁰GLP-2(1-33);
Arg³⁰Lys³⁴GLP-2(1-34);
Arg³⁰Lys³⁵GLP-2(1-35);
Arg^{30,35}Lys²⁰GLP-2(1-35);
Arg³⁵GLP-2(1-35).

In another preferred embodiment, the parent peptide is Lys²⁰GLP-2(1-33) or Lys²⁰Arg³⁰GLP-2(1-33).

In another preferred embodiment, the parent peptide is Arg³⁰Lys³⁴GLP-2(1-34).

In another preferred embodiment, the parent peptide is Arg³⁰Lys³⁵GLP-2(1-35); Arg^{30,35}Lys²⁰GLP-2(1-35) or Arg³⁵GLP-2(1-35).

In another preferred embodiment, the parent peptide is GLP-2(1-35) or an analogue thereof.

In a further preferred embodiment, the C-terminal amino acid residue is present in the form of the amide.

The present invention also relates to a composition comprising a variant of the GLP-2 peptide. The variant is one in which one or more amino acid residues have been substituted by other amino acid residues. In a particularly preferred embodiment Ala has been substituted by Gly in position 2 of the mature peptide. It is expected that this variant will exhibit a longer plasma half-life than the native peptide, which is an advantage because the dosage required to obtain an adequate appetite-suppressing or satiety-inducing effect will generally be smaller.

In a particular aspect, the invention relates to use of a pharmaceutical composition comprising a peptide with the following amino acid sequence

(SEQ ID NO:1)

X¹ H X² D G S F S D E M N T X³ L D X⁴ L A X⁵ X⁶ D
F I N W L X⁷ X⁸ T K I T D X⁹

wherein

X¹ is NH₂, DFPEEVAIVEELGRR (SEQ ID NO:2), DFPEEVTIVEELGRR (SEQ ID NO:3), DFPEEVNIVEELRRR (SEQ ID NO:4), or a fragment thereof,

X² is Ala or Gly,

X³ is Ile or Val,

X⁴ is Asn, Ser or H is,

X⁵ is Ala or Thr,

X⁶ is Arg or Lys,

X⁷ is Ile or Leu,

X⁸ is Gln or H is, or

X⁹ is OH, Lys, Arg, Arg-Lys, Lys-Arg, Arg-Arg or Lys-Lys

for the prophylaxis or treatment of diseases or disorders associated with impaired appetite regulation.

GLP-2 Derivatives

The term “derivative” is defined herein as a peptide in which one or more of the amino acid residues of a parent peptide have been chemically modified, e.g. by alkylation, acylation, ester formation, or amide formation.

The term “GLP-2 derivative” is defined herein as a derivative of GLP-2 or an analogue and/or fragment thereof. The parent peptide from which such a derivative is formally derived is in some places referred to as the “GLP-2 moiety” of the derivative.

Lipophilic Substituent

To obtain a satisfactory protracted profile of action, a lipophilic substituent is attached to the GLP-2 moiety. The lipophilic substituent preferably comprises 4-40 carbon atoms, in particular 8-25 carbon atoms.

Preferably, the GLP-2 derivatives of the present invention have one or two lipophilic substituents. In a most preferred embodiment, the GLP-2 derivatives of the present invention have one lipophilic substituent.

The lipophilic substituent may be attached to an amino group of the GLP-2 moiety by means of a carboxyl group of the lipophilic substituent which forms an amide bond with an amino group of the amino acid to which it is attached. As an alternative, the lipophilic substituent may be attached to said amino acid in such a way that an amino group of the lipophilic substituent forms an amide bond with a carboxyl group of the amino acid. As a further option, the lipophilic substituent may be linked to the GLP-2 moiety via an ester bond. Formally, the ester can be formed either by reaction between a carboxyl group of the GLP-2 moiety and a hydroxyl group of the substituent-to-be or by reaction between a hydroxyl group of the GLP-2 moiety and a carboxyl group of the substituent-to-be. As a further alternative, the lipophilic substituent can be an alkyl group which is introduced into a primary amino group of the GLP-2 moiety.

The lipophilic substituent may be attached to any one amino acid residue. However, if a lipophilic is attached to the N-terminal or C-terminal amino acid residue of the parent peptide, the lipophilic substituent must be an ω-carboxylic acid group or an alkyl group.

In a preferred embodiment, the lipophilic substituent is attached to any one of the amino acid residues in positions 20-34, preferably 30-34, most preferably 30.

In another preferred embodiment of the present invention, the lipophilic substituent has a group which can be negatively charged. One preferred such group is a carboxylic acid group.

In a further preferred embodiment, the lipophilic substituent is attached to an amino acid residue in such a way that a carboxyl group of the lipophilic substituent forms an amide bond with an amino group of the amino acid residue.

In a further preferred embodiment, the lipophilic substituent is attached to an amino acid residue in such a way that an amino group of the lipophilic substituent forms an amide bond with a carboxyl group of the amino acid residue.

In a further preferred embodiment, the lipophilic substituent comprises a partially or completely hydrogenated cyclopentanophenathrene skeleton.

In a further preferred embodiment, the lipophilic substituent is a straight-chain or branched alkyl group.

In a further preferred embodiment, the lipophilic substituent is the acyl group of a straight-chain or branched fatty acid.

In a further preferred embodiment, the lipophilic substituent is an acyl group selected from the group comprising CH₃(CH₂)ₙCO—, wherein n is 4 to 38, preferably CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In a further preferred embodiment, the lipophilic substituent is an acyl group of a straight-chain or branched alkane α,ω-dicarboxylic acid.

In a further preferred embodiment, the lipophilic substituent is attached to the parent peptide by means of a spacer. For example, the lipophilic substituent may be attached to the GLP-2 moiety by means of a spacer in such a way that a carboxyl group of the spacer forms an amide bond with an amino group of the GLP-2 moiety.

Examples of suitable spacers are succinic acid, Lys, Glu or Asp, or a dipeptide such as Gly-Lys. When the spacer is succinic acid, one carboxyl group thereof may form an amide bond with an amino group of the amino acid residue, and the other carboxyl group thereof may form an amide bond with an amino group of the lipophilic substituent. When the spacer is Lys, Glu or Asp, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the ε-amino group of Lys and the lipophilic substituent. In one preferred embodiment, such a further spacer is succinic acid which forms an amide bond with the ε-amino group of Lys and with an amino group present in the lipophilic substituent. In another preferred embodiment such a further spacer is Glu or Asp which forms an amide bond with the ε-amino group of Lys and another amide bond with a carboxyl group present in the lipophilic substituent, that is, the lipophilic substituent is an N^{ε}-acylated lysine residue.

In a further preferred embodiment, the spacer is an unbranched alkane α,ω-dicarboxylic acid group having from 1 to 7 methylene groups, preferably two methylene groups which spacer forms a bridge between an amino group of the parent peptide and an amino group of the lipophilic substituent.

In a further preferred embodiment, the spacer is an amino acid residue except Cys, or a dipeptide such as Gly-Lys or any unbranched alkane α,ω-amino acid having from 1 to 7 methylene groups, preferably 2-4 methylene groups, which form a bridge between an amino group of the parent peptide and an amino group of the lipophilic substituent. The phrase “a dipeptide such as Gly-Lys” is used to designate a dipeptide wherein the C-terminal amino acid residue is Lys, H is or Trp, preferably Lys, and wherein the N-terminal amino acid residue is selected from the group comprising Ala, Arg, Asp, Asn, Gly, Glu, Gln, Ile, Leu, Val, Phe, and Pro.

In a further preferred embodiment, the lipophilic substituent is attached to the parent peptide by means of a spacer which is an amino acid residue except Cys, or is a dipeptide such as Gly-Lys and wherein a carboxyl group of the parent peptide forms an amide bond with an amino group of a Lys residue or a dipeptide containing a Lys residue, and the other amino group of the Lys residue or a dipeptide containing a Lys residue forms an amide bond with a carboxyl group of the lipophilic substituent.

In a further preferred embodiment, the lipophilic substituent is attached to the parent peptide by means of a spacer which is an amino acid residue except Cys, or is a dipeptide such as Gly-Lys and wherein an amino group of the parent peptide forms an amide bond with a carboxylic group of the amino acid or dipeptide spacer, and an amino group of the amino acid or dipeptide spacer forms an amide bond with a carboxyl group of the lipophilic substituent.

In a further preferred embodiment, the spacer is an amino acid residue except Cys, or is a dipeptide such as Gly-Lys and wherein a carboxyl group of the parent peptide forms an amide bond with an amino group of the amino acid or dipeptide spacer, and the carboxyl group of the amino acid or dipeptide spacer forms an amide bond with an amino group of the lipophilic substituent.

In a further preferred embodiment, the spacer is an amino acid residue except Cys, or is a dipeptide such as Gly-Lys, and wherein a carboxyl group of the parent peptide forms an amide bond with an amino group of Asp or Glu, or a dipeptide containing an Asp or Glu residue, and a carboxyl group of the spacer forms an amide bond with an amino group of the lipophilic substituent.

In a further preferred embodiment, the lipophilic substituent is an acyl group selected from the group comprising HOOC(CH₂)ₘCO—, wherein m is 4 to 38, preferably HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

In a further preferred embodiment, the lipophilic substituent is a group of the formula CH₃(CH₂)ₚ((CH₂)_{q}COOH)CHNH—CO(CH₂)₂CO—, wherein p and q are integers and p+q is an integer of from 8 to 40, preferably from 12 to 35.

In a further preferred embodiment, the lipophilic substituent is a group of the formula CH₃(CH₂)ᵣCO_NHCH(COOH)(CH₂)₂CO⁻, wherein r is an integer of from 10 to 24.

In a further preferred embodiment, the lipophilic substituent is a group of the formula CH₃(CH₂)SCO—NHCH((CH₂)₂COOH)CO—, wherein s is an integer of from 8 to 24.

In a further preferred embodiment, the lipophilic substituent is a group of the formula COOH(CH₂)ₜCO— wherein t is an integer of from 8 to 24.

In a further preferred embodiment, the lipophilic substituent is a group of the formula —NHCH(COOH)(CH₂)₄NH—CO(CH₂)ᵤCH₃, wherein u is an integer of from 8 to 18.

In a further preferred embodiment, the lipophilic substituent is a group of the formula —NHCH(COOH)(CH₂)₄NH—COCH((CH₂)₂COOH)NH—CO(CH₂)ₙCH₃, wherein w is an integer of from 10 to 16.

In a further preferred embodiment, the lipophilic substituent is a group of the formula —NHCH(COOH)(CH₂)₄NH—CO(CH₂)₂CH(COOH)NH—CO(CH₂)_{w}CH₃, wherein x is an integer of from 10 to 16.

In a further preferred embodiment, the lipophilic substituent is a group of the formula —NHCH(COOH)(CH₂)₄NH—CO(CH₂)₂CH(COOH)NHCO(CH₂)_{y}CH₃, wherein y is zero or an integer of from 1 to 22.

Preferred GLP-2 Derivatives

Preferred GLP-2 derivatives of the present invention are:

Lys²⁰(N^{ε}-tetradecanoyl)GLP-2(1-33);

Lys^{20,30}-bis(N″-tetradecanoyl)GLP-2(1-33);

Lys²⁰(N^{ε}-tetradecanoyl)Arg³⁰GLP-2(1-33);

Lys²⁰(N^{ε}-tetradecanoyl)Arg³⁰GLP-2(1-33);

Arg³⁰Lys³⁵(N^{ε}-tetradecanoyl)GLP-2(1-35);

Arg^{30,35}Lys²⁰(N^{ε}-tetradecanoyl)GLP-2(1-35);

Arg³⁵Lys³⁰(N^{ε}-tetradecanoyl)GLP-2(1-35);

Arg³⁰Lys³⁴(N^{ε}-tetradecanoyl)GLP-2(1-34);

Lys²⁰(N^{ε}-(ε-carboxynonadecanoyl))GLP-2(1-33);

Lys^{20,30}-bis(N^{ε}-(ε-carboxynonadecanoyl))GLP-2(1-33);

Lys²⁰(N^{ε}-(ω-carboxynonadecanoyl))Arg³⁰GLP-2(1-33);

Arg³⁰Lys³⁵(N^{ε}(ω-carboxynonadecanoyl))GLP-2(1-35);

Arg^{30,35}Lys²⁰(N^{ε}-(ω-carboxynonadecanoyl))GLP-2(1-35);

Arg³⁵Lys³⁰(N^{ε}-(ω-carboxynonadecanoyl))GLP-2(1-35); and

Arg³⁰Lys³⁴(N^{ε}-(ω-carboxynonadecanoyl))GLP-2(1-34).

Pharmaceutical Compositions

The present invention also relates to pharmaceutical compositions comprising a GLP-2 derivative of the invention. In a preferred embodiment, the pharmaceutical compositions are provided in the form of a composition suitable for administration by injection. Such a composition can either be an injectable solution ready for use or it can be an amount of a solid composition, e.g. a lyophilized product, which has to be dissolved in a solvent before it can be injected

In a preferred embodiment, the concentration of the GLP-2 derivative in the pharmaceutical compositions of the present invention is not less than 0.5 mg/ml, preferably not less than about 5 mg/ml, more preferably not less than about 10 mg/ml and, most preferably, not more than about 100 mg/ml.

The pharmaceutical composition of the present invention preferably further comprise one or more of the following substances:
a pharmaceutically acceptable vehicle or carrier;
an isotonic agent, preferably selected from the group consisting of sodium chloride, mannitol, and glycerol;
a preservative, preferably selected from the group consisting of phenol, m-cresol, methyl p-hydroxybenzoate, butyl p-hydroxybenzoate and benzyl alcohol;
a buffer, preferably selected from the group consisting of sodium acetate, citrate, glycylglycine, histidine, 2-phenylethanol and sodium phosphate; and
a surfactant capable of improving the solubility and/or the stability of the GLP-2 derivative, preferable selected from poloxymer 188, tween 20 and tween 80.

Further to the above-mentioned components, solutions containing a GLP-2 derivative of the present invention may also contain a surfactant in order to improve the solubility and/or the stability of the derivative.

The present invention also relates to pharmaceutical compositions comprising a GLP-2 derivative which has a helix content as measured by CD at 222 nm in H₂O at 22±2° C. exceeding 25%, preferably in the range of 25% to 50%, at a peptide concentration of about 10 μM. The size of the partially helical, micelle-like aggregates may be estimated by size-exclusion chromatography. Similarly, the apparent (critical micelle concentrations) CMC's of the peptides may be estimated from the concentration dependent fluorescence in the presence of appropriate dyes (e.g. Brito, R. & Vaz, W. (1986) Anal. Biochem. 152, 250-255).

That the derivatives have a partially structured micellar-like aggregate conformation in aqueous solutions makes them more soluble and stable in solution over a wide concentration range as compared to the native peptide. The increased solubility and stability can be seen by comparing the solubility after 9 days of standing for a derivative and native GLP-2(1-34) in a pharmaceutical formulation, e.g. 5 mM phosphate buffer, pH 6.9 added 0.1 M NaCl.

Circular Dichroism (CD) can be used to show that the GLP-2 derivatives have a certain partially structured conformation independent of their concentration. In contrast, for native GLP-2 an increase in the helix content is seen with increasing concentration, from 10-15% to 30-35% (at 500 μM concentration) in parallel with peptide self-association. For the GLP-2 derivatives forming partially structured micellar-like aggregates in aqueous solution the helix content remains constant above 30% at concentrations of 10 μM. The aggregated structured conformation is an inherent property of the derivative present in water or dilute aqueous buffer without the need for any additional structure-inducing components. Note that the CD signal is proportional to the average content of α-helix in the peptides, i.e., a CD value of −1 corresponds to 10% α-helix content under these conditions.

The pharmaceutical compositions of the present invention may be prepared by conventional techniques, e.g. as described in Remington's Pharmaceutical Sciences, 1985 or in Remington: The Science and Practice of Pharmacy, 19^{th} edition, 1995. For example, the injectable compositions can be prepared using the conventional techniques of the pharmaceutical industry which involves dissolving and mixing the ingredients as appropriate to give the desired end product.

The composition may be in a form suited for systemic injection or infusion and may, as such, be formulated with a suitable liquid vehicle such as sterile water or an isotonic saline or glucose solution. The compositions may be sterilized by conventional sterilization techniques which are well known in the art. The resulting aqueous solutions may be packaged for use or filtered under aseptic conditions and lyophilized, the lyophilized preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

The pharmaceutical composition of the present invention may also be adapted for nasal, transdermal, pulmonal, or rectal administration. The pharmaceutically acceptable carrier or diluent employed in the composition may be any conventional solid carrier. Examples of solid carriers are lactose, terra alba, sucrose, talc, gelatin, agar, pectin, acacia, magnesium stearate and stearic acid. Similarly, the carrier or diluent may include any sustained release material known in the art, such as glyceryl monostearate or glyceryl distearate, alone or mixed with a wax.

It may be of particular advantage to provide the composition of the invention in the form of a sustained release formulation. As such, the composition may be formulated as microcapsules or microparticles containing the GLP-2 peptide encapsulated by or dispersed in a suitable pharmaceutically acceptable biodegradable polymer such as polylactic acid, polyglycolic acid or a lactic acid/glycolic acid copolymer.

For nasal administration, the preparation may contain GLP-2 peptide dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Generally, the compounds of the present invention are dispensed in unit dosage form comprising 0.5-500 mg of the peptide together with a pharmaceutically acceptable carrier per unit dosage.

According to one procedure, the GLP-2 derivative is dissolved in an amount of water which is somewhat less than the final volume of the composition to be prepared. An isotonic agent, a preservative and a buffer is added as required and the pH value of the solution is adjusted—if necessary—using an acid, e.g. hydrochloric acid, or a base, e.g. aqueous sodium hydroxide as needed. Finally, the volume of the solution is adjusted with water to give the desired concentration of the ingredients.

A composition for nasal administration of GLP-2 may, for example, be prepared as described in European Patent No. 272097 (to Novo Nordisk A/S) or in WO 93/18785.

Uses

The GLP-2 derivatives of the present invention can be used in the treatment of various diseases, including obesity, small bowel syndrome, Crohn's disease, ileitis, intestinal inflammation, gastric and duodenal ulceration, inflammatory bowel disease (IBD) and intestinal cancer damage therapy. The particular GLP-2 derivative to be used and the optimal dose level for any patient will depend on the disease to be treated and on a variety of factors including the efficacy of the specific peptide derivative employed, the age, body weight, physical activity, and diet of the patient, on a possible combination with other drugs, and on the severity of the case. It is recommended that the dosage of the GLP-2 derivative of this invention be determined for each individual patient by those skilled in the art.

The pharmacological properties of the compounds of the invention can be tested e.g. as described in our International Patent Application No. PCT/DK97/00086 the content of which is hereby incorporated in its entirety by reference.

The GLP-2 derivatives may be administered parenterally to patients in need of such a treatment. Parenteral administration may be performed by subcutaneous, intramuscular, or intravenous injection by means of a syringe, optionally a pen-like syringe. Alternatively, parenteral administration can be performed by means of an infusion pump. A further option is a composition which may be a powder or a liquid for the administration of the GLP-2 derivative in the form of a nasal or pulmonal spray. As a still further option, the GLP-2 derivatives of the invention can also be administered transdermally, e.g. from a patch, optionally an iontophoretic patch, or transmucosally, e.g. bucally.

Methods of Production

The parent peptide can be produced by a method which comprises culturing a host cell containing a DNA sequence encoding the peptide and capable of expressing the peptide in a suitable nutrient medium under conditions permitting the expression of the peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection).

The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of peptide in question.

The DNA sequence encoding the parent peptide may suitably be of genomic or cDNA origin, for instance obtained by preparing a genomic or cDNA library and screening for DNA sequences coding for all or part of the peptide by hybridization using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the peptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The DNA sequence may also be prepared by polymerase chain reaction using specific primers, for instance as described in U.S. Pat. No. 4,683,202 or Saiki et al., Science 239 (1988), 487-491.

The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the peptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art, cf. for instance Sambrook et al., supra.

The DNA sequence encoding the peptide may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracycline chloramphenicol, neomycin, hygromycin, or methotrexate.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present peptide and includes bacteria, yeast, fungi, and higher eukaryotic cells. Examples of suitable host cells well known and used in the art are, without limitation, E. coli, Saccharomyces cerevisiae, or mammalian BHK or CHO cell lines.

The GLP-2 derivatives of the invention can be prepared by introducing the lipophilic substituent into the parent GLP-2 or GLP-2 analogue using methods known per se, see for example WO 95/07931, the contents of which is hereby incorporated in its entirety by reference.

N-acylation of a Lys residue can be carried out by using an activated amide of the acyl group to be introduced as the acylating agent, e.g. the amide with benzotriazole. The acylation is carried out in a polar solvent in the presence of a base.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

The following acronyms for commercially available chemicals are used:
NMP N-Methyl-2-pyrrolidone.
EDPA: N-Ethyl-N,N-diisopropylamine.
TFA: Trifluoroacetic acid.
Myr-ONSu: Tetradecanoic acid 2,5-dioxopyrrolidin-1-yl ester.
   Abbreviations:
PDMS: Plasma Desorption Mass Spectrometry
HPLC: High Performance Liquid Chromatography
amu: atomic mass units

### Example 1

### Synthesis of Lys³⁰(N^{ε}-tetradecanoyl) hGLP-2

A mixture of hGLP-2 (10.0 mg, 2.7 μmol), EDPA (9.6 mg, 74.3 μmol), NMP (210 μl) and water (100 μl) was gently shaken for 15 min. at room temperature. To the resulting mixture was added a solution of Myr-ONSu (21.5 mg, 6.6 μmol) in NMP (32 μl). The reaction mixture was gently shaken for 30 min. at room temperature, and an additional amount of a solution of Myr-ONSu (14.4 mg, 4.4 μmol) in NMP (22 μl). The resulting mixture was gently shaken for 15 min. at room temperature. The reaction was quenched by the addition of a solution of glycine (4.5 mg, 4.5 μmol) in 50% aqueous ethanol (451 μl). The reaction mixture was purified by column chromatography using a cyanopropyl column (Zorbax 300SB-CN) and a standard acetonitrile/TFA system. The column was heated to 65° C. and the acetonitrile gradient was 0-100% in 60 minutes. The title compound (5.0 mg, 47%) was isolated from the eluate.

### Example 2

### Synthesis of Lys³⁰ (N^{ε}-Cy-glutamyl(N^{O}-tetradecanoyl))) hGLP-2

To a mixture of hGLP-2-OH (5 mg, 1.33 μmol), EDPA (4.8 mg, 37.2 μmol), NMP (0.7 ml) and water (0.35 ml) was added a solution of Myr-Glu(ONSu)-OBu^{t} (2 mg, 4 μmol), prepared as described in PCT application no. PCT/DK97/00340, in NMP (51 μl). The reaction mixture was gently shaken for 5 min., and then allowed to stand for an additional 110 min. at room temperature. The reaction was quenched by the addition of a solution of glycine (2.2 mg, 29.3 μmol) in water (22 μl). A 0.5% aqueous solution of ammonium acetate (15 ml) was added, and the resulting mixture eluted onto a Varian 5 g C8 Mega Bond Elut®, the immobilized compound washed with 5% aqueous acetonitrile (20 ml), and finally liberated from the cartridge by elution with TFA (20 ml). The eluate was concentrated in vacuo, and the residue purified by column chromatography using a cyanopropyl column (Zorbax 300SB-CN) and a standard acetonitrile/TFA system. The column was heated to 65° C. and the acetonitril gradient was 0-100% in 60 minutes. The title compound (0.1 mg, 1.8%) was isolated, and the product was analyzed by PDMS. The m/z value for the protonated molecular ion was found to 26276 4107.8±3. The resulting molecular weight is thus 4106.8±3 amu (theoretical value 4106 amu).

### Additional Aspects

1. A GLP-2 derivative comprising a lipophilic substituent attached to any one amino acid residue.

2. A GLP-2 derivative of aspect 1 with the proviso that only if the substituent has an ω-carboxylic acid group or is an alkyl group can it be attached to the N-terminal or C-terminal amino acid residue of the parent peptide.

3. A GLP-2 derivative of aspect 1 or 2, wherein the lipophilic substituent comprises from 4 to 40 carbon atoms, more preferred from 8 to 25.

4. A GLP-2 derivative of any of the preceding aspects, wherein said lipophilic substituent is attached to said amino acid in such a way that a carboxyl group of the lipophilic substituent forms an amide bond with an amino group of the amino acid.

5. A GLP-2 derivative of any of aspects 1-3, wherein said lipophilic substituent is attached to said amino acid in such a way that an amino group of the lipophilic substituent forms an amide bond with a carboxyl group of the amino acid.

6. A GLP-2 derivative of any of the preceding aspects, wherein the lipophilic substituent is attached to the parent peptide by means of a spacer.

7. A GLP-2 derivative of aspect 6, wherein the spacer is an unbranched alkane α,ω-dicarboxylic acid group having from 1 to 7 methylene groups, preferably two methylene groups which form a bridge between an amino group of the parent peptide and an amino group of the lipophilic substituent.

8. A GLP-2 derivative of aspect 6, wherein the spacer is an amino acid residue except Cys, or a dipeptide such as Gly-Lys.

9. A GLP-2 derivative of aspect 8, wherein a carboxyl group of the parent peptide forms an amide bond with an amino group of Lys or a dipeptide containing a Lys residue, and the other amino group of the Lys or a dipeptide containing a Lys residue forms an amide bond with a carboxyl group of the lipophilic substituent.

10. A GLP-2 derivative of aspect 8, wherein an amino group of the parent peptide forms an amide bond with a carboxylic group of the amino acid or dipeptide spacer, and an amino group of the amino acid or dipeptide spacer forms an amide bond with a carboxyl group of the lipophilic substituent.

11. A GLP-2 derivative of aspect 8, wherein a carboxyl group of the parent peptide forms an amide bond with an amino group of the amino acid or dipeptide spacer, and the carboxyl group of the amino acid or dipeptide spacer forms an amide bond with an amino group of the lipophilic substituent.

12. A GLP-2 derivative of aspect 8, wherein a carboxyl group of the parent peptide forms an amide bond with an amino group of Asp or Glu, or a dipeptide containing an Asp or Glu residue, and a carboxyl group of the spacer forms an amide bond with an amino group of the lipophilic substituent.

13. A GLP-2 derivative of any of the preceding aspects, wherein the lipophilic substituent comprises a partially or completely hydrogenated cyclopentanophenathrene skeleton.

14. A GLP-2 derivative of any of aspects 1-12, wherein the lipophilic substituent is an straight-chain or branched alkyl group.

15. A GLP-2 derivative of any of aspects 1-12, wherein the lipophilic substituent is the acyl group of a straight-chain or branched fatty acid.

16. A GLP-2 derivative of aspect 15 wherein the acyl group is selected from the group comprising CH₃(CH₂)ₙCO—, wherein n is 4 to 38, preferably CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

17. A GLP-2 derivative of any of aspects 1-12, wherein the lipophilic substituent is an acyl group of a straight-chain or branched alkane α,ω-dicarboxylic acid.

18. A GLP-2 derivative of aspect 17 wherein the acyl group is selected from the group comprising HOOC(CH₂)ₘCO—, wherein m is 4 to 38, preferably HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

19. A GLP-2 derivative of any of aspects 1-12, wherein the lipophilic substituent is a group of the formula CH₃(CH₂)ₚ((CH₂)_{q}COOH)CHNH—CO(CH₂)₂CO—, wherein p and q are integers and p+q is an integer of from 8 to 40, preferably from 12 to 35.

20. A GLP-2 derivative of any of aspects 1-12, wherein the lipophilic substituent is a group of the formula CH₃(CH₂)ᵣCO—NHCH(COOH)(CH₂)₂CO—, wherein r is an integer of from 10 to 24.

21. A GLP-2 derivative of any of aspects 1-12, wherein the lipophilic substituent is a group of the formula CH₃(CH₂)ₛCO—NHCH((CH₂)₂COOH)CO—, wherein s is an integer of from 8 to 24.

22. A GLP-2 derivative of any of aspects 1-12, wherein the lipophilic substituent is a group of the formula COOH(CH₂)ₜCO— wherein t is an integer of from 8 to 24.

23. A GLP-2 derivative of any of aspects 1-12, wherein the lipophilic substituent is a group of the formula —NHCH(COOH)(CH₂)₄NH—CO(CH₂)ᵤCH₃, wherein u is an integer of from 8 to 18.

24. A GLP-2 derivative of any of aspects 1-12, wherein the lipophilic substituent is a group of the formula —NHCH(COOH)(CH₂)₄NH—COCH((CH₂)₂COOH)NH—CO(CH₂)ₙCH₃, wherein w is an integer of from 10 to 16.

25. A GLP-2 derivative of any of aspects 1-12, wherein the lipophilic substituent is a group of the formula —NHCH(COOH)(CH₂)₄NH—CO(CH₂)₂CH(COOH)NH—CO(CH₂)ₙCH₃, wherein x is an integer of from 10 to 16.

26. A GLP-2 derivative of any of aspects 1-12, wherein the lipophilic substituent is a group of the formula —NHCH(COOH)(CH₂)₄NH—CO(CH₂)₂CH(COOH)NHCO(CH₂)_{y}CH₃, wherein y is zero or an integer of from 1 to 22.

27. A GLP-2 derivative of any of the preceding aspects which has one lipophilic substituent.

28. A GLP-2 derivative of any of aspects 1-26 which has two lipophilic substituents.

29. A GLP-2 derivative according any of the preceding aspects, wherein the parent peptide is selected from the group comprising GLP-2(1-30); GLP-2(1-31); GLP-2(1-32); GLP-2(1-33); GLP-2(1-34) and GLP-2(1-35) or an analogue or a fragment thereof.

30. A GLP-2 derivative of aspect 29, wherein the parent peptide is selected from the group comprising GLP-2(1-35) or an analogue or a fragment thereof.

31. A GLP-2 derivative of aspect 29 or 30 wherein the designation analogue comprises derivatives wherein a total of up to ten amino acid residues have been exchanged with any α-amino acid residue.

32. A GLP-2 derivative of any of the preceding aspects wherein the parent peptide is selected from the group comprising Lys²⁰GLP-2(1-33); Lys²⁰Arg³⁰GLP-2(1-33); Arg³⁰Lys³⁵GLP-2(1-35); Arg^{30,35}Lys²⁰GLP-2(1-35); Arg³⁵GLP-2(1-35); Arg³⁰Lys³⁴GLP-2(1-34).

33. A GLP-2 derivative of aspect 1 selected from the group consisting of

Lys²⁰(N^{ε}-tetradecanoyl)GLP-2(1-33);
Lys^{20,30}-bis(N″-tetradecanoyl)GLP-2(1-33);
Lys²⁰(N^{ε}-tetradecanoyl)Arg³⁰GLP-2(1-33);
Lys²⁰(N^{ε}-tetradecanoyl)Arg³⁰GLP-2(1-33);
Arg³⁰Lys³⁵(N^{ε}-tetradecanoyl)GLP-2(1-35);
Arg^{30,35}Lys²⁰(N^{ε}-tetradecanoyl)GLP-2(1-35);
Arg³⁵Lys³⁰(N^{ε}-tetradecanoyl)GLP-2(1-35);
Arg³⁰Lys³⁴(N^{ε}-tetradecanoyl)GLP-2(1-34);
Lys²⁰(N^{ε}-(ω-carboxynonadecanoyl))GLP-2(1-33);
Lys^{20,30}-bis(N″-(ω-carboxynonadecanoyl))GLP-2(1-33);
Lys²⁰(N^{ε}-(ω-carboxynonadecanoyl))Arg³⁰GLP-2(1-33);
Arg³⁰Lys³⁵(N^{ε}-(ω-carboxynonadecanoyl))GLP-2(1-35);
Arg^{30,35}Lys²⁰(N^{ε}-(ω-carboxynonadecanoyl))GLP-2(1-35);
Arg³⁵Lys³⁰(N^{ε}-(ω-carboxynonadecanoyl))GLP-2(1-35); and
Arg³⁰Lys³⁴(N^{ε}-(ω-carboxynonadecanoyl))GLP-2(1-34).
   34. A pharmaceutical composition comprising a GLP-2 derivative of any of the preceding aspects and a pharmaceutically acceptable vehicle or carrier.
   35. A method of treating obesity in a patient in need of such a treatment, comprising administering to the patient a therapeutically effective amount of a GLP-2 derivative of any of aspects 1-33 together with a pharmaceutically acceptable carrier.
   36. A method of treating small bowel syndrome in a patient in need of such a treatment, comprising administering to the patient a therapeutically effective amount of a GLP-2 derivative of any of aspects 1-33 together with a pharmaceutically acceptable carrier.
   37. A pharmaceutical composition comprising a GLP-2 derivative which has a helix content as measured by CD at 222 nm in H₂O at 22±2° C. exceeding 25%, preferably in the range of 25% to 500%, at a peptide concentration of about 10 μM.
   38. A pharmaceutical composition of aspect 37, wherein the concentration of GLP-2 derivative is not less than 0.5 mg/ml, preferably not less than about 5 mg/ml, more preferred not less than about 10 mg/ml and, preferably, not more than about 100 mg/ml.
   39. A pharmaceutical composition of aspect 37 or 38, comprising a GLP-2 derivative wherein at least one amino acid residue of the parent peptide has a lipophilic substituent attached.
   40. A pharmaceutical composition of aspect 39, comprising a GLP-2 derivative having a lipophilic substituent which is attached to any one of the amino acid residues in position 20-34, preferably 30-34, most preferably 30.
   41. A pharmaceutical composition of any of aspects 37-40, further comprising a pharmaceutically acceptable vehicle or carrier.
   42. A pharmaceutical composition of any of aspects 37-41, further comprising an isotonic agent, preferably selected from the group consisting of sodium chloride, mannitol and glycerol.
   43. A pharmaceutical composition of any of aspects 37-42, further comprising a preservative, preferably selected from the group consisting of phenol, m-cresol, methyl p-hydroxybenzoate, butyl p-hydroxybenzoate and benzyl alcohol.
   44. A pharmaceutical composition of any of aspects 37-43, further comprising a buffer, preferably selected from the group consisting of sodium acetate, citrate, glycylglycine, histidine, 2-phenylethanol and sodium phosphate.
   45. A pharmaceutical composition of any of aspects 37-44, further comprising a surfactant capable of improving the solubility and/or the stability of the GLP-2 derivative, preferable selected from poloxymer 188, tween 20 and tween 80.
   46. A pharmaceutical composition of any of aspects 37-45, wherein the parent peptide is selected from the group comprising GLP-2(1-30); GLP-2(1-31); GLP-2(1-32); GLP-2(1-33); GLP-2(1-34) and GLP-2(1-35).
   47. A pharmaceutical composition of any of aspects 37-46, wherein the parent peptide has the following amino acid sequence (SEQ ID NO: 1) X¹H X² D G S F S D E M N T X³ L D X⁴ L A X⁵ X⁶ D F 1 N W L X⁷ X⁸ T K I T D X⁹
   wherein
X¹ is NH₂, DFPEEVAIVEELGRR (SEQ ID NO:2), DFPEEVTIVEELGRR (SEQ ID NO:3), DFPEEVNIVEELRRR (SEQ ID NO:4), or a fragment thereof,
X² is Ala or Gly,
X³ is Ile or Val,
X⁴ is Asn, Ser or H is,
X⁵ is Ala or Thr,
X⁶ is Arg or Lys,
X⁷ is Ile or Leu,
X⁸ is Gln or H is, and
X⁹ is OH, Lys, Arg, Arg-Lys, Lys-Arg, Arg-Arg or Lys-Lys.
   48. A pharmaceutical composition of any of aspects 37-47, comprising a GLP-2 derivative wherein a total of up to fifteen, preferably up to ten, more preferably up to six, amino acid residues have been exchanged with any α-amino acid residue which can be coded for by the genetic code.
   49. A pharmaceutical composition of any of aspects 37-48, wherein the parent peptide is selected from the group comprising Lys²⁰GLP-2(1-33); Lys²⁰Arg³⁰GLP-2(1-33); Arg³⁰Lys³⁴GLP-2(1-34); Arg³⁰Lys³⁵GLP-2(1-35); Arg^{30,35}Lys²⁰GLP-2(1-35); Arg³⁵GLP-2(1-35).
   50. A method for improving the solubility and/or stability of GLP-2 or a fragment or an analogue thereof, comprising introducing a lipophilic substituent on any one of the amino acid residues of the parent peptide.
   51. A method of aspect 50, wherein a lipophilic substituent is introduced on any one of the amino acid residues in position 20-34, preferably 30-34, most preferred 30.
   52. A method of aspect 50 or 51, wherein the lipophilic substituent comprises from 4 to 40 carbon atoms, preferably from 8 to 25 carbon atoms.

53. A method of any of aspects 50 to 52, wherein the lipophilic substituent is the acyl group of a straight-chain or branched fatty acid.

54. A method of aspect 53, wherein the acyl group is selected from the group comprising CH₃(CH₂)ₙCO—, wherein n is 4 to 38, preferably CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

55. A method of any of aspects 50 to 54, wherein the parent peptide is selected from the group comprising Lys²⁰GLP-2(1-33); Lys²⁰Arg³⁰GLP-2(1-33); Arg³⁰Lys³⁴GLP-2(1-34); Arg³⁰Lys³⁵GLP-2(1-35); Arg^{30,35}Lys²⁰GLP-2(1-35); Arg³⁵GLP-2(1-35).

56. A method for treating obesity, comprising administering to a subject in need thereof a pharmaceutical composition of any of aspects 37 to 49.

57. A method for treating small bowel syndrome, Crohn's disease, ileitis, intestinal inflammation, gastric and duodenal ulceration, inflammatory bowel disease (IBD) and intestinal cancer damage therapy, comprising administering to a subject in need thereof a pharmaceutical composition of any of aspects 37 to 49.

58. Use of a pharmaceutical composition comprising a peptide with the following amino acid sequence

X1 H X2 D G S F S D E M N T X3 L D X4 L A X5 X6 D F I N W L X7 X8 T K I T D X9 wherein X1 is NH2, DFPEEVAIVEELGRR, DFPEEVTIVEELGRR, DFPEEVNIVEELRRR, or a fragment thereof,

X² is Ala or Gly,

X³ is Ile or Val,

X⁴ is Asn, Ser or H is,

X⁵ is Ala or Thr,

X⁶ is Arg or Lys,

X⁷ is lie or Leu,

X⁸ is Gin or H is, and

X⁹ is OH, Lys, Arg, Arg-Lys, Lys-Arg, Arg-Arg or Lys-Lys

together with a pharmaceutically acceptable excipient or vehicle for appetite suppression or sateity induction.

59. The use of a composition according to aspect 59, wherein X¹ is NH2.

60. The use of a composition according to aspect 59, wherein X² is Ala.

61. The use of a composition according to aspect 59, wherein X³ is Ile.

62. The use of a composition according to aspect 59, wherein X⁴ is Asn.

63. The use of a composition according to aspect 59, wherein X⁵ is Ala.

64. The use of a composition according to aspect 59, wherein X⁶ is Arg.

65. The use of a composition according to aspect 59, wherein X⁷ is Ile.

66. The use of a composition according to aspect 59, wherein X⁸ is Gln.

67. The use of a composition according to aspect 59, wherein X⁹ is OH.

68. The use of a composition according to aspect 59, wherein the peptide has the sequence

(SEQ ID NO:5)

HADGSFSDEMNTILDNLAARDFIQTKITD,

(SEQ ID NO:6)

HADGSFSDEMNTILDNLATRDFINWLIQTKITD,
or

(SEQ ID NO:7)

HADGSFSDEMNTVLDNLATRDFINWLLHTKITD.

69. The use of a composition according to any of aspects 59-71, for the prophylaxis or treatment of diseases or disorders associated with impaired appetite regulation.
70. The use of a composition according to any of the aspects 59-70 for the prophylaxis or treatment of obesity or type II diabetes.
71. A pharmaceutical composition comprising a peptide of any of aspects 59-70 in combination with another appetite-suppressing or satiety-inducing agent.
72. A composition according to aspect 73, wherein said other appetite suppressing or satiety-inducing agent is glucagon-likepeptide-1.
73. A method of treating diseases or disorders associated with impaired appetite regulation, the method comprising administering to an individual in need of such treatment an amount of a peptide according to any of aspects 59-70 sufficient to suppress appetite or induce satiety in said individual.
74. A method according to aspect 83, wherein the disease or disorder is obesity or type II diabetes.
75. A method according to aspect 83, wherein the amount of the peptide is in the range of from about 10 llg/kg body weight to about 5 mg/kg body weight.
76. A method of treating diseases or disorders associated with impaired appetite regulation, the method comprising administering to an individual in need of such treatment an amount of a peptide according to aspect 59 sufficient to suppress appetite or induce satiety in said individual.
77. A method according to aspect 86, wherein the disease or disorder is obesity or type II diabetes.
78. A method according to aspect 86, wherein the amount of the peptide is in the range of from about 10 pg/kg body weight to about 5 mg/kg body weight.
79. A method of treating diseases or disorders associated with impaired appetite regulation, the method comprising administering to an individual in need of such treatment an amount of a fraction according to aspect 86 sufficient to suppress appetite or induce satiety in said individual.
80. A method according to aspect 89, wherein the disease or disorder is obesity or type II diabetes.
81. Use of a peptide according to any of aspects 59-70 for the manufacture of a medicament for the prophylaxis or treatment of diseases or disorders associated with impaired appetite regulation.

What is claimed is:

1. A composition comprising (a) a non-naturally occurring polypeptide having an amino acid sequence according to the formula X1 H X2 D G S F S D E M N T X3 L D X4 L A X5 X6 D F I N W L X7 X8 T K I T D X9 (SEQ ID NO: 1) and (b) a pharmaceutically acceptable combination of (i) an isotonic agent, (ii) a buffer, and (iii) a preservative, a surfactant, or a combination of a surfactant and a preservative,
wherein (I) X1 is NH₂, DFPEEVAIVEELGRR (SEQ ID NO:2), DFPEEVTIVEELGRR (SEQ ID NO:3), DFPEEVNIVEELRRR (SEQ ID NO:4), or a fragment of any of SEQ ID NOS:2-4; X² is Ala or Gly; X³ is Ile or Val; X⁴ is Asn, Ser, or H is; X⁵ is Ala or Thr; X⁶ is Arg or Lys; X⁷ is Ile or Leu; X⁸ is Gln or H is; and X⁹ is OH, Lys, Arg, Arg-Lys, Lys-Arg, Arg-Arg, or Lys-Lys and (II) the solubility of the peptide, stability of the peptide, or both is significantly greater than the solubility and/or stability of the peptide without the combination.

2. The composition of claim 1, wherein the composition comprises a preservative.

3. The composition of claim 2, wherein X2 is Gly.

4. A method of promoting the treatment of small bowel syndrome, Crohn's disease, ileitis, intestinal inflammation, gastric ulceration, duodenal ulceration, inflammatory bowel disease, or intestinal cancer damage therapy in a patient comprising administering an effective amount of the composition of claim 2 to the patient.

5. The method of claim 4, wherein the method comprises administering an effective amount of the composition of claim 3 to the patient.

6. A non-naturally occurring peptide having an amino acid sequence according to the formula X1 H X2 D G S F S D E M N T X3 L D X4 LA X5 X6 D F I N W L X7 X8 T K I T D X9 (SEQ ID NO: 1),
wherein X1 is NH₂, DFPEEVAIVEELGRR (SEQ ID NO:2), DFPEEVTIVEELGRR (SEQ ID NO:3), DFPEEVNIVEELRRR (SEQ ID NO:4), or a fragment of any of SEQ ID NOS:2-4; X2 is Ala or Gly; X3 is Ile or Val; X4 is Asn, Ser, or H is; X5 is Ala or Thr; X6 is Arg or Lys; X7 is Ile or Leu; X8 is Gln or H is; and X9 is OH, Lys, Arg, Arg-Lys, Lys-Arg, Arg-Arg, or Lys-Lys.

7. The non-naturally occurring peptide of claim 6, wherein X2 is Gly.

8. A lyophilized composition comprising the non-naturally occurring peptide of claim 6.

9. The composition of claim 6, wherein X² of the peptide is Gly.

10. A method of promoting the treatment of small bowel syndrome, Crohn's disease, ileitis, intestinal inflammation, gastric ulceration, duodenal ulceration, inflammatory bowel disease, or intestinal cancer damage therapy in a patient comprising administering an effective amount of the peptide of claim 6 to the patient.

11. The method of claim 10, wherein X2 of the peptide is Gly.


SEQUENCE LISTING (7 sequences)

SEQ ID NO: 1 - protein, 35 aa, Artificial
  Synthetic
  MISC_FEATURE (2)..(2): Xaa=Ala or Gly
  MISC_FEATURE (13)..(13): Xaa=Ile or Val
  MISC_FEATURE (16)..(16): Xaa=Asn,Ser or His
  MISC_FEATURE (19)..(19): Xaa=Ala or Thr
  MISC_FEATURE (20)..(20): Xaa=Arg or Lys
  MISC_FEATURE (27)..(27): Xaa=Ile or Leu
  MISC_FEATURE (28)..(28): Xaa=Gln or His
  MISC_FEATURE (34)..(34): Xaa=OH, Lys or Arg
  MISC_FEATURE (35)..(35): Xaa=Lys,Arg or is missing
HXDGSFSDEMNTXLDXLAXXDFINWLXXTKITDXX

SEQ ID NO: 2 - protein, 15 aa, Artificial
  Synthetic
DFPEEVAIVEELGRR

SEQ ID NO: 3 - protein, 15 aa, Artificial
  Synthetic
DFPEEVTIVEELGRR

SEQ ID NO: 4 - protein, 15 aa, Artificial
  Synthetic
DFPEEVNIVEELRRR

SEQ ID NO: 5 - protein, 29 aa, Artificial
  Synthetic
HADGSFSDEMNTILDNLAARDFIQTKITD

SEQ ID NO: 6 - protein, 33 aa, Artificial
  Synthetic
HADGSFSDEMNTILDNLATRDFINWLIQTKITD

SEQ ID NO: 7 - protein, 33 aa, Artificial
  Synthetic
HADGSFSDEMNTVLDNLATRDFINWLLHTKITD